(19) United States
(12) Patent Application Publication
(10) Pub. No.: US 2011/0052024 A1
(43) Pub. Date: Mar. 3, 2011

(54) METHOD OF ANALYSING STROKE IMAGES

(76) Inventor: Wieslaw Lucjan Nowinski, Singapore (SG)

(21) Appl. No.: 12/676,677

(22) PCT Filed: Aug. 18, 2008
(86) PCT No.: PCT/SG08/00303

§ 371 Date: Jul. 1, 2010

Related U.S. Application Data
(60) Provisional application No. 60/935,936, filed on Sep. 7, 2007.

(51) Int. Cl. G06K 9/00; A61B 5/00

(52) U.S. Cl. 382/131; 600/300

(57) ABSTRACT

A plurality of brain atlases 1, 2, 3 are co-registered 4, and mapped 8 to a scan of a brain. The mapping is then tested 9 to determine the presence of a stroke, and whether it is ischemic or hemorrhagic. This provides an accurate way of identifying strokes using patient data which is particularly suitable for use in an emergency situation such as the emergency department of a hospital.

### FIELD OF THE INVENTION

The present invention relates to a system, having method and apparatus aspects, for analysing brain scan images of a patient suspected of having a stroke, particularly a system which is of use in the emergency department (ED) of a hospital.

### BACKGROUND OF THE INVENTION

The speed and efficiency of patient evaluation are critical factors in an emergency department. The key questions to be answered are whether 1) a patient has suffered a stroke or not, and if so whether 2) the stoke is ischemic or hemorrhagic.

### SUMMARY OF THE INVENTION

The present invention aims to provide a new and useful methods and systems to determine from a scan whether a patient is suffering from a stroke.

In general terms the invention proposes that a plurality of brain atlases are co-registered, and mapped to a scan of a brain, and then the mapping is used to determine the presence of a stroke.

Preferred embodiments of the invention provide an accurate way of identifying strokes from patient brain scan data (such as CT or MRI data), and one which is particularly suitable for use in an emergency situation such as the emergency department of a hospital.

The invention may be expressed as a method of processing brain scans, as an apparatus for doing so, or as a computer program product storing software operable by a computer system to perform the method.

### BRIEF DESCRIPTION OF THE EMBODIMENTS

An embodiment of the invention will now be described, for sake of example only, with reference to the accompanying drawings, in which:

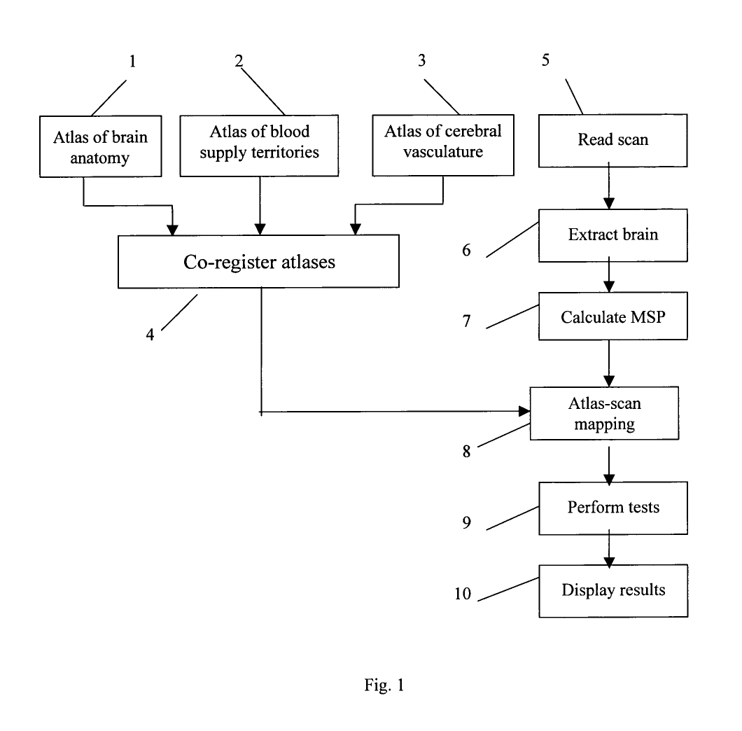

FIG. 1 is a flow diagram of an embodiment of the invention; and

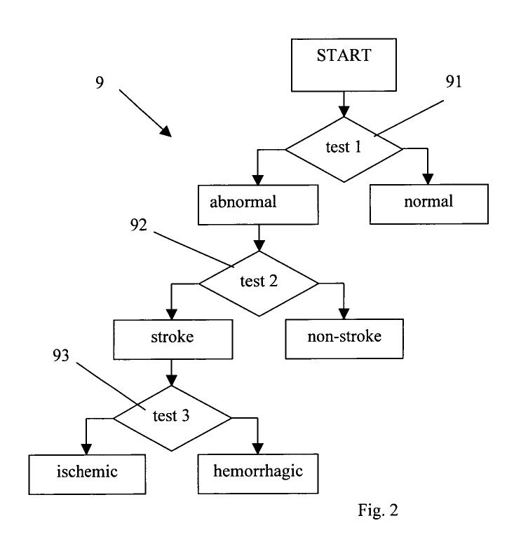

FIG. 2 is a flow diagram of the decision tree used in one of the steps of FIG. 1.

### DETAILED DESCRIPTION OF THE EMBODIMENTS

Referring to FIG. 1, the flow diagram of an embodiment of the invention is shown. The embodiment is a method which may be performed by any conventional computer system, for example a computer system having a processor which is caused to follow the method by reading operating instructions from a computer program product, such as a recording medium storing the instructions.

The embodiment employs three input data sets which are respective brain atlases:
an atlas of brain anatomy 1
an atlas of cerebral vasculature 2
and an atlas of blood supply territories 3.

Any existing brain atlases can be used for this purpose, for instance the atlases developed in our lab [1][2][3][4]. The atlases are fully segmented and labeled, and their 3D version constructed.

In a first step 4 of the embodiment, all the atlases 1, 2, 3 are mutually co-registered using any existing techniques, for instance the FTT approach [5]. In an alternative form of the method, the co-registration process is performed in advance, and data representing the three atlases and their co-registration is input to the computer system, for example on the computer program product mentioned above.

The other input to the method is scan data 5 obtained by one or more scans of a particular patient.

In a step 6, the skull and scalp as well as any other extra-cerebral objects are removed from the scan. For this purpose, any existing method can be used, for instance that disclosed in [8] [10].

In a step 7, the midsagittal plane is extracted from the scan. For this purpose, any existing method can be used, for instance that disclosed in [6] [7]. In an alternative form of the embodiment, which improves accuracy, particularly, for the brains with a curved interhemispheric fissure, step 7 is replaced with a step of obtaining the midsagittal lines for each slice as calculated, for example in [11].

In a step 8, the mutually co-registered atlases obtained in step 4 are mapped into the scan. For this purpose any existing method can be used, for instance the FTT [5] or the statistical-based approach [9]. In addition, warping against ventricles can be used, particularly, for elder people with prominent vascular dilation. For ventricular extraction, the method described in [12] can be used. For atlas warping against the ventricles, the method described in [13] and [14] can be employed.

The mapping between the segmented and labeled brain atlases delineate regions of interest in the scan data. Any set of regions of interests with anatomical structures, vessels, and/or blood supply territories can be identified and used for analysis in the following steps of the embodiment.

In a step 9, a series of tests 91, 92, 93 applied to the output of step 9, in order to make reach a view in relation to the test. The decision tree is shown in FIG. 2.

When performing tests 91, 92, 93, all regions of interest identified using the atlases, or any subset of them, are compared. The comparison can be done by comparing corresponding identified regions of interest in the left and right hemispheres individually (i.e. one to one) or for any group of regions of the same patient, and/or by comparing the identified regions of interest to data obtained from normal patients.

The first test 91 is to determine whether the image contains asymmetry. The comparison can employ statistics of various kinds, in particular, the mean values and standard deviations (e.g. by obtaining values for these for each hemisphere, and declaring asymmetry if they differ by more than a predetermined threshold), as well as other standard statistical tests available in SPSS eg. [15]. More advanced techniques to capture asymmetry can also be applied including [16] [17] [18].

Statistical testing can be combined with image processing techniques to eliminate certain unwanted features from the image. In particular, low and high intensity thresholds can be set manually eliminating certain image regions (i.e. the ones outside the range between these thresholds), so that ventricles and/or the skull can be removed, optionally the images may be smoothed initially by performing median or anisotropic smoothing, and then the statistical tests can applied to the intensities within the defined range [19] [20] [21] [22] [23] [24] [25][26] [27]. In principle the smoothing step can be useful even if the thresholds are not employed.

The scan is considered normal if all the corresponding regions tested produce no significant difference. If any region varies significantly from that in the contralateral hemisphere (or normal), the scan is considered abnormal.

The second test 92 is to determine whether the asymmetry is due to a stroke, or instead to some other factor. There are several situations mimicking the stroke, and additional acquisitions and human intervention may be necessary to distinguish stroke from no stroke pathology [28], [29].

Sub-step 93 is discrimination between ischemic and hemorrhagic scans. This can be done based on intensity distribution [28] and [29]. In CT scans, hyperdensity signals hemorrahage, while hypointensity indicates ischemia. On T2 MR scans, this relationship is the reverse. Hounsfield Units (HU) can further be used for discrimination; for instance, HU range of 60-100 corresponds to blood.

In a final step 10, the results of step 9 are output.

### REFERENCES

1. Nowinski W L, Thirunavuukarasuu A, Bryan R N: The Cerefy Atlas of Brain Anatomy. An Introduction to Reading Radiological Scans for Students, Teachers, and Researchers. Thieme, New York, 2002.
2. Nowinski, W. L., A, Thirunavuukarasuu., Volkau. I., Baimuratov, R., Hu, Q., Aziz, A., Huang, S., 2005. Three-dimensional atlas of the brain anatomy and vasculature. RadioGraphics, 25(1):263-71.
3. Nowinski W L, Qian G, Bhanu Prakash K N, Thirunavuukarasuu A, Hu Q M, Ivanov N, Parimal A S, Runge V L, Beauchamp N J: Analysis of ischemic stroke MR images by means of brain atlases of anatomy and blood supply territories. Academic Radiology 2006; 13(8):1025-1034.
4. Nowinski W L, Thirunavuukarasuu A, Volkau, Marchenko Y, Runge V M: The Cerefy Atlas of Cerebral Vasculature. Thieme, New York, 2008.
5. Nowinski W L, Qian G, Bhanu Prakash K N, Hu Q, Aziz A: Fast Talairach Transformation for magnetic resonance neuroimages. Journal of Computer Assisted Tomography 2006; 30(4):629-41.
6. Nowinski W L, Bhanu Prakash K N, Volkau I, Ananthasubramaniam A, Beauchamp N J: Rapid and automatic calculation of the midsagittal plane in magnetic resonance diffusion and perfusion images. Academic Radiology, 13(5) 2006:652-663.
7. Volkau I, Bhanu Prakash K. N., Anand A, Aziz A, Nowinski W L: Extraction of the midsagittal plane from morphological neuroimages using the Kullback-Leibler's measure. Medical Image Analysis 2006, 10(6): 863-874.
8. Hu Q, Qian G, Nowinski W L: Adaptive brain segmentation from T1-weighted and SPGR MR scans. BIL/Z/04154 submitted on 9 Feb. 2006. BIL/P/04154/00/US, Provisional application No. 60/781,736 filed 14 Mar. 2006.
9. Volkau I, Bhanu Prakash K N, Ng T T, Gupta V, Nowinski W L: Localization of the anterior and posterior commissures on low quality images based on geometrical fitting of brain and statistical analysis. BIL/Z/04234, BIL/P/04287/00/US, Provisional application no filed on 24 Aug. 2006.
10. Shattuck D W, Sandor-Leahy S R, Schaper K A, Rottenberg D A, Leahy R M: Magnetic Resonance Image tissue classification using a partial volume model. Neuroimage, 13(5):856-876.
11. Hu Q, Nowinski W L: A rapid algorithm for robust and automatic extraction of the midsagittal plane of the human cerebrum from neuroimages based on local symmetry and outlier removal. NeuroImage 2003; 20(4):2154-2166.
12. Nowinski W L, Xia Y, Aziz A, Hu Q M: Method and apparatus for extracting cerebral ventricular system from images. PCT/SG03/00043, PCT application filed on 27 Feb. 2003; WO2004/0077359 published on 10 Sep. 2004.
13. Nowinski W L, Qian G Y, Hu Q, K N Bhanuprakash, Ivanov N, Huang S: Fast and automatic interpretation of normal morphological brain scans by using an atlas with non-linear warping. Program 91th Radiological Society of North America Scientific Assembly and Annual Meeting RSNA 2005, Chicago, Ill., USA, 27 Nov.-2 Dec. 2005:857.
14. Ivanov N, Parimal A S, Nowinski W L: Method and program for non-linear image warping based on specific class of radial functions. BIL/Z/00936; BIL/P//2405/PCT filed 14 Dec. 2005.
15. SPSS15.0 Manuals CD-ROM: www.spss.com
16. Volkau I, Nowinski W L: A method and apparatus for determining asymmetry in an image, BIL//P/1833/SG, SG 200405043-1 filed on 10 Sep. 2004. BIL/P/1833/2837/PCT-PCT/SG2005/000302 filed on 1 Sep. 2005. WO2006/028416 published on 16 Mar. 2006. (former invention title: Information based method for identification and localization of pathology in MRI Neuroimages)
17. Nowinski W L, Hu Q: Method and apparatus for identifying pathology in brain images, BIL/P//1226/PCT, PCT/SG03/00284 filed on 12 Dec. 2003. WO2005/057498 published on 23 Jun. 2005. BIL/P/1226/3257/EP, 03781267.4 filed on 12 Jun. 2006.
18. Gupta V, Bhanu Prakash K N, Nowinski W L: Automatic identification of infarct slices and hemisphere from DWI scans. BIL/P/04489/00/US, U.S. 60/273,019 Provisional application filed on 6 Dec. 2006.
19. Sahoo P K, Soltani S, Wong A K C: A survey of thresholding techniques, Comput. Vision Graphics Image Process 1988; 41: 233-260.
20. Fu K S, Mui J K: A survey on image segmentation. Pattern Recognition 1981, 13(1): 3-16.
21. Weszka J S: A survey of threshold selection techniques. Computer Graphics and Image Processing 1978; 7:259-265.
22. M. Sezgin, B. Sankur, Survey over image thresholding techniques and quantitative performance evaluation, J. Electron. Imaging 2004, 13(1): 146-165.
23. Hildebrandt K, Polthier K: Anisotropic filtering of non-linear surface features. Computer Graphics Forum, 2004, 23(3):391-400.

24. Gerig G, Kubler O, Kikinis R, Jolesz F A: Nonlinear anisotropic filtering of MRI data. IEEE Trans Med Imaging 1992, 11: 221-232.

25. Seramani S, Zhou J, Chan K L, Malmurugan N, Nagappan A: Denoising of MR Images using non linear anisotropic diffusion filtering as a preprocessing step. International Journal of BioSciences and Technology, IJBST 2008, 1(1):17-21.
26. Manjón J V, Carbonell-Caballero J, Lull J J, Garcia-Martí G, Marti-Bonmatí L, Robles M: MRI denoising using non-local means. Medical Image Analysis 2008, 12:514-523.
27. Toprak A, Özerdem M S, Gïler Suppression of impulse noise in MR images using artificial intelligent based neuro-fuzzy adaptive median filter. Digital Signal Processing 2008, 18:391-405
28. Gonzalez R G, Hirsch J A, Koroshetz W J, Lev M H, Schaefer P: Acute Ischemic Stroke: Imaging and Intervention. Springer; 1 edition 2005.
29. Mohr J P, Grotta J C, Choi D W, Wolf P A: Stroke: Pathophysiology, Diagnosis, and Management (4th edition), Churchill Livingstone, 2004.

1. An apparatus for identifying a stroke using brain scan data of a patient, the apparatus comprising a processor arranged to perform the steps of:
forming a mapping between the scan data and a plurality of co-registered brain atlases;
identifying a plurality of regions of interest in the scan data using the co-registered brain atlases; and
testing one or more of the identified regions of interest, thereby identifying the presence of a stroke.

2. An apparatus according to claim 1 in which the co-registered brain atlases comprise one or more of:
an atlas of brain anatomy;
an atlas of cerebral vasculature; and
an atlas of blood supply territories.

3. An apparatus according to claim 1 in which the processor is arranged to co-register the brain atlases.

4. An apparatus according to claim 1 in which said testing comprises identifying whether an asymmetry exists between corresponding regions of interest in different hemispheres of the brain, and, if so, identify abnormality.

5. An apparatus according to claim 4 in which said testing further comprises identifying the presence of a stroke.

6. An apparatus according to claim 5 in which said testing further comprises identifying whether the stroke is ischemic or hemorrhagic.

7. An apparatus according to claim 1 in which, prior forming said mapping, the brain scan is subject to a process for extracting the portions of the image which represent background.

8. An apparatus according to claim 1 in which, prior to forming said mapping, a mid-sagittal plane is extracted, the mid-sagittal plane being used in the mapping process.

9. A method for identifying a stroke using brain scan data of a patient, the method comprising:
forming a mapping between the scan data and a plurality of co-registered brain atlases;
identifying a plurality of regions of interest in the scan data using the co-registered brain atlases; and
testing one or more of the identified regions of interest, thereby identifying the presence of a stroke.

10. A method according to claim 9 in which the co-registered brain atlases comprise one or more of:
an atlas of brain anatomy;
an atlas of cerebral vasculature; and
an atlas of blood supply territories.

11. A method according to claim 9 further comprising co-registering the brain atlases.

12. A method according claim 9 in which said testing comprises identifying whether an asymmetry exists between corresponding regions of interest in different hemispheres of the brain, and, if so, identifying abnormality

13. A method according to claim 12 in which said testing further comprises identifying the presence of a stroke.

14. A method according to claim 13 in which said testing further comprises identifying whether the stroke is ischemic or hemorrhagic.

15. A method according to claim 9 in which, prior forming said mapping, the brain scan is subject to a process for extracting the portions of the image which represent background.

16. A method according to claim 9 in which, prior to forming said mapping, a mid-sagittal plane is extracted, the mid-sagittal plane being used in said step of forming said mapping.
